(19) United States
(12) Patent Application Publication
(10) Pub. No.: US 2007/0112309 A1
(43) Pub. Date: May 17, 2007

(54) Withdrawal syringe

(76) Inventor: Jerry Zucker, Charleston, SC (US)

Correspondence Address: HAMMER & HANF, PC, 3125 SPRINGBANK LANE, SUITE G, CHARLOTTE, NC, 28226, US

(21) Appl. No.: 11/281,573

(22) Filed: Nov. 17, 2005

(51) Int. Cl. A61M 5/315

(52) U.S. Cl. 604/227

(57) ABSTRACT

A withdrawal syringe has a cannula connected to one end of a receiving cylinder having a filter in the other end. A cup shaped piston is in a sliding telescoping relation with the cylinder. The piston has finger grips and a thumb rest is secured from the cylinder in spaced opposition to the finger grips. The syringe is operated by one hand by a squeezing action between the fingers and thumb.

### BACKGROUND OF THE INVENTION

This invention relates to syringes and more particularly to a device for withdrawal of liquids from the body.

Under conventional practices, blood or other body liquids are withdrawn by connecting a needle to a pre-evacuated container or by a manually operated syringe. Presently available systems are not easily operated with precision with one hand. The withdrawal procedure with evacuated systems may cause collapse of the vein, especially in older people. In many mechanical systems, the forces which are exerted on the piston create reaction forces tending to make the withdrawal procedure awkward.

### SUMMARY OF THE INVENTION

In accordance with the present invention, a withdrawal syringe comprises a needle or cannula connected to one end of a receiving cylinder having a filter element at the other end. A cup-shaped piston is provided around the cylinder and is movable in telescoping relation with the cylinder. A ring seal is provided between the two parts. Finger flanges extend from the piston, and a thumb rest is provided beyond the finger flanges and is connected by legs back to the cylinder. Friction or gear engagement may be provided between the legs and the outer surfaces of the piston.

The device is operated by pulling on the piston with the first two fingers with the thumb on the thumb rest. This allows for a very precise insertion of the needle and precise, controlled withdrawal of liquid with the force of the thumb opposing the force of the fingers.

### THE DRAWINGS

FIG. 1 is a perspective assembly view of the syringe of the present invention.

FIG. 2 is a partial longitudinal sectional view of the assembled syringe of FIG. 1.

FIG. 3 is an enlarged view of the indicated portion of FIG. 2.

FIG. 4 is a modification of the features shown in FIG. 3.

### DESCRIPTION OF THE PREFERRED EMBODIMENTS

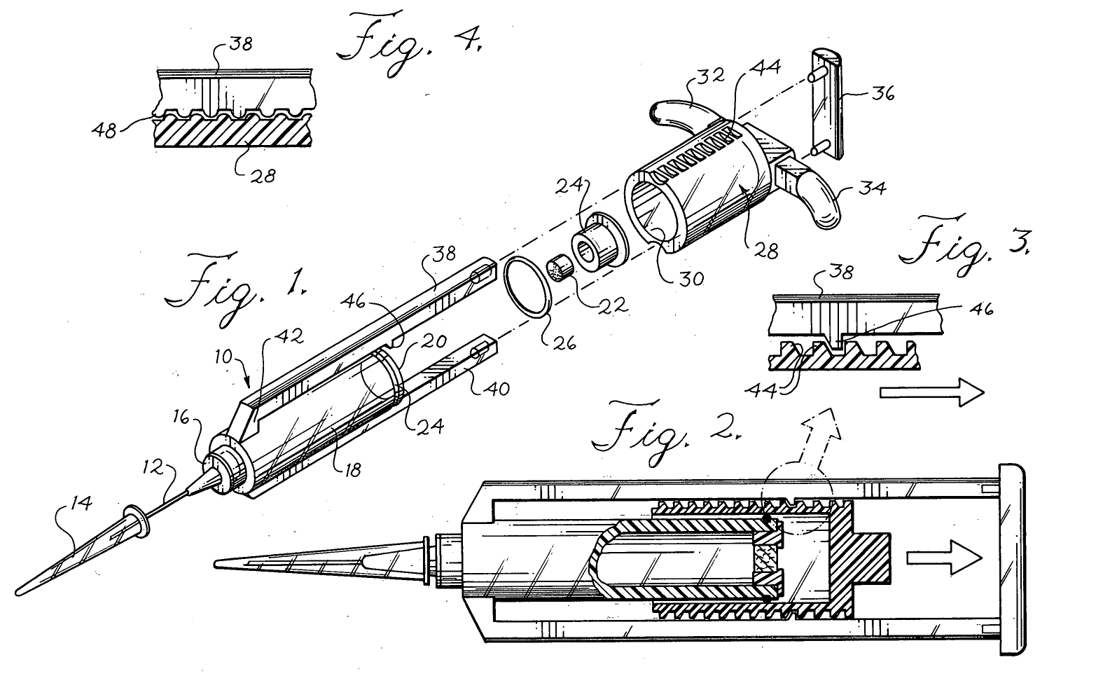

FIGS. 1 and 2 show an elongated syringe 10 having a hollow needle or cannula 12 extending from one end, with the needle being protected by a removable sheath 14. The needle is connected to one end 16 of a hollow receiving cylinder 18. The other end 20 of cylinder 18 is provided with a microporous filter 22 retained in a cap 24 covering the cylinder end 20. The filter 22 allows for transmission of gas or air into and out of the cylinder 18 without contamination of the cylinder interior. A flexible O-ring seal 26 is provided in a circular groove 27 formed in the outer wall of cylinder 18 near the filter end 20 thereof.

A cup-shaped piston 28 having a cylindrical inner surface 30 and an open and closed end is provided in telescoping coaxial relation with the cylinder 18 and in sliding sealing engagement with the seal 26. It may be seen that when the piston 28 is moved in a direction away from the needle 12 as shown by the arrow in FIG. 2, pressure is reduced in the cylinder.

A pair of forwardly curved flanges or legs 32 and 34 extend in opposite directions from the closed end of the piston 28, in directions generally perpendicular to the longitudinal axis of the syringe. A thumb rest 36 is provided at or beyond the outer extent of travel of the piston 28. The thumb rest 36 extends generally perpendicular to the flanges 32-34 and is secured by means of pins or the like to one end of a pair of spaced parallel legs 38 and 40. The other end of the legs are bent inwardly at 42 and are secured to the outer wall of the cylinder 18 near the forward end 16 thereof.

It may be seen that the distance between the facing surfaces of the legs 38 and 40 is somewhat greater than the outer diameter of the piston 28 to allow movement of the piston in an axial direction relative to the cylinder 18 and thumb rest. The device is operated by grasping the flanges 32-34 with the first two fingers with the thumb of the same hand resting on the thumb rest 36. A balanced squeezing action is used to withdraw liquids through the cannula 12.

To provide a more precise feel and control some degree of interaction may be provided between an outer surface of the piston 28 and the inner surface of the legs 38 and 40. For example, as shown in FIGS. 1, 2 and 3, the outer surface of the piston 28 may be provided with an axially extending track having a plurality of spaced teeth 44, with an inwardly facing cog 46 on the legs in locking engagement with successive teeth as the piston is withdrawn. If desired, the teeth may be shaped to provide a ratchet as shown, allowing only outward movement of the piston. Also, the piston may be later rotated to bring the teeth out of engagement with the cog, allowing ejection of the liquid.

As shown in FIG. 4, the surfaces between a leg 38 and the cylinder 28 may be simply roughened as shown at 48 to provide some degree of resistance to motion and improved control.

1. A withdrawal syringe comprising a cylinder having opposite ends, a cannula operatively connected to one end of the cylinder, a cup-shaped piston in movable telescoping relation around the cylinder, means for providing a sealing relation between said cylinder and piston, finger grip means secured to said piston, and thumb support means extending from said cylinder in opposition to said finger grip means, said finger grip and thumb support means being operable with one hand to move the piston away from the cannula.

2. The syringe of claim 1 wherein said thumb support means is secured to one end of an elongate member, the other end of said elongate member being secured to said cylinder.

3. The syringe of claim 1 additionally comprising means for providing a degree of resistance to movement of said piston.

4. The syringe of claim 3 wherein said means for providing a degree of friction comprises ratchet means allowing movement of the piston in only one direction.

5. The syringe of claim 1 wherein the finger grip means comprises a pair of flanges secured to and extending in opposite directions from the piston.

6. The syringe of claim 1 wherein a gas permeable filter is provided in the other end of the cylinder.
